(19) United States
(12) Patent Application Publication
(10) Pub. No.: US 2014/0296792 A1
(43) Pub. Date: Oct. 2, 2014

(54) SHARPS GUARD

(71) Applicant: Thomas Chun, Northridge, CA (US)

(72) Inventor: Thomas Chun, Northridge, CA (US)

(21) Appl. No.: 13/853,907

(22) Filed: Mar. 29, 2013

(51) Int. Cl. A61M 5/32

(52) U.S. Cl. CPC A61M 5/3216
USPC 604/192; 604/263

(57) ABSTRACT

An arrangement for covering the sharp point of a needle of a syringe or similar needle device. The arrangement includes a hollow cover which exposes the needle in a retracted position and covers the needle in the deployed condition. A resilient multi-section link anchored to the syringe may propel the hollow cover to the deployed position when the link or the needle device is pressed against an environmental surface. The arrangement may be integral with the needle device or may be formed as a separate component which is attachable to the needle device. An optional cap may cover the needle when the hollow cover is in the retracted position.

### FIELD OF THE INVENTION

The present invention relates to protective devices for so-called medical and other “sharps” such as hypodermic needles and syringes. More particularly, the protective device is a cover which covers the exposed sharp point, and which snaps into operative position upon being pressed from the side of the syringe.

### BACKGROUND OF THE INVENTION

Hypodermic needles, syringes, autoinjectors, and like devices have long presented medical hazards to their users. This may include patients using such devices themselves, but also medical caregivers. The sharp point of a medical needle easily pierces human tissues, and upon doing so, may for example transfer contaminants including infectious materials such as contaminated blood to an otherwise healthy person. This is a well-known hazard which has received considerable attention in the prior art.

Prior art devices for covering the sharp point of a medical needle have been proposed. However, many known protective devices suffer from various deficiencies including unduly complicated and therefore expensive construction. They may require tedious assembly and use by a person well versed in their operation. There exists a need for a practical, uncomplicated guard device for medical sharps and like devices which may be utilized for purposes other than medical.

### SUMMARY OF THE INVENTION

The present invention addresses the above stated need by providing a sharps cover or guard for a sharp point which may be formed in a single piece, and may be deployed without obliging the user to grasp the needle or any portion of a syringe or autoinjector close to the dangerous sharp point. Moreover, the novel guard may be deployed by pressing its associated needle device laterally against a solid surface such as a desk or table top. The guard may project into the deployed position covering the point very fast in what may be called snap action.

The novel sharps guard may be fabricated inexpensively from a suitable synthetic polymer. The novel sharps guard may be provided as a stand-alone device which is installed on a needle device, or alternatively, may be formed as part of the needle device.

It is an object of the invention to provide a sharps guard which deploys without obliging a user to grasp the needle or to have his or her fingers in close proximity to the sharp point of the needle.

Another object of the invention is to enable deployment by pressing the needle device laterally against a solid surface.

It is an object of the invention to provide improved elements and arrangements thereof by apparatus for the purposes described which is inexpensive, dependable, and fully effective in accomplishing its intended purposes.

These and other objects of the present invention will become readily apparent upon further review of the following specification and drawings.

### BRIEF DESCRIPTION OF THE DRAWINGS

Various objects, features, and attendant advantages of the present invention will become more fully appreciated as the same becomes better understood when considered in conjunction with the accompanying drawings, in which like reference characters designate the same or similar parts throughout the several views, and wherein:

FIG. 1 is a partial side view of a needle device having a protective guide according to at least one aspect of the invention, showing the guard in a retracted position.

FIG. 2 is similar to FIG. 1, but with one component deleted from the view and showing the guard in its operational or deployed position.

FIG. 3 is similar to FIG. 2, but showing the guard moved to a slightly different position.

FIG. 4 is a side view of a guard according to a further aspect of the invention, and is drawn to scale greater than that of FIGS. 1-3.

FIG. 5 is a front perspective detail view taken from near the right side of FIG. 2, and looking from the right towards the left.

### DETAILED DESCRIPTION

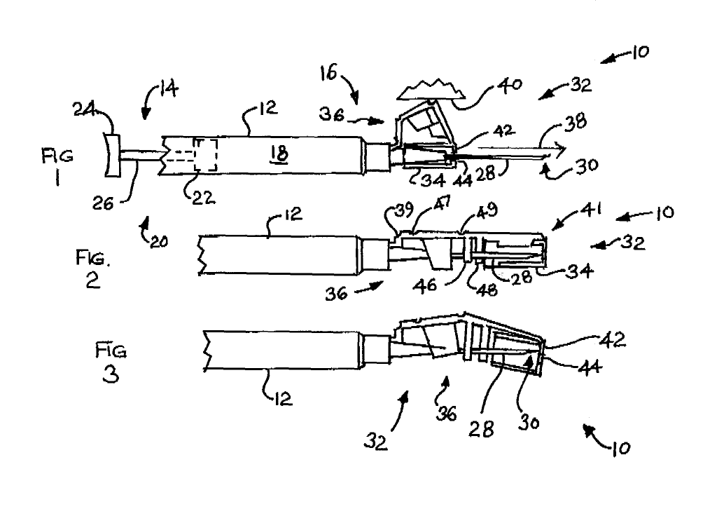

Referring first to FIG. 1, there is shown according to at least one aspect of the invention a needle device 10 for containing a supply of a fluid (not shown), penetrating a substrate (not shown), and establishing fluid communication between the needle device 10 and the substrate for transfer of fluids therebetween. It is contemplated that one function of the needle device 10 is delivery of a liquid into a substrate such as human or animal bodily tissue. However, it should be recognized that the present invention may be used in delivery of fluids, which may include gases, into other substrates. Also, the reverse is true. That is, fluids may be withdrawn from a substrate and received by the needle device 10. An example is taking blood samples from patients, although as is also true for delivery of fluids, uses other than medical are contemplated.

The needle device 10 may comprise conventional fluid handling apparatus, including at a minimum a hollow housing 12 having a proximal end 14, a distal end 16, and a fluid chamber 18 disposed between the proximal end 14 and the distal end 16. A piston assembly 20 is slidably disposed within the fluid chamber 18, and may include a piston 22 disposed to exert force on fluid contained within the fluid chamber 18. The piston 22 may be operated by a pressure head 24 which is disposed to receive manual force from a person using the needle device 10. The pressure head 24 may be connected to the piston 22 by a shaft 26. That end of the fluid chamber 18 located proximate the distal end 16 of the needle device 10 secures a hollow needle 28 which disposed in fluid communication with the fluid chamber 18. The fluid chamber 18 will be understood to be suitably sealed at the proximal end 14 and the distal end 16 to prevent undesired escape of fluid to be received or injected. The hollow needle 28 has a longitudinal axis and terminates in a sharp point 30 capable of penetrating the substrate. As is typical in medical usages, the substrate is tissue of a live human or animal. Of course, fluids may be injected into or received from other objects using a needle device such as the needle device 10. As the components described thus far may be conventional, they are not fully shown or described in full detail.

The needle device 10 may include a guard 32 for covering the sharp point 30 of the needle 28. The guard 32 may comprise a hollow cover 34 for covering the sharp point 30. The hollow cover 34 may be cylindrical for example, and is adapted to be movable between a deployed position covering the sharp point 30 (shown in FIG. 2) and a retracted position (shown in FIG. 1) which leaves the sharp point 30 sufficiently exposed to be able to penetrate the substrate. An arrow 38 indicates direction of motion of the hollow cover 34 when moving to the deployed position.

A spring mechanism 36 is disposed to propel the hollow cover 34 from the retracted position to the deployed position when the guard 32 is urged against an environmental object in a direction which is out of registry with the longitudinal axis of the needle 28 such that force is imposed on the spring mechanism 36. In FIG. 1, should the needle device 10 be urged or pressed upwardly against an exemplary environmental surface 40, forces will be exerted on the spring mechanism 36 which cause rapid deployment of the hollow cover 34, or alternatively stated, cause the hollow cover 34 to move in the direction of the arrow 38 to the deployed position seen in FIG. 2.

Specifically referring to FIG. 2, it will be seen that the spring mechanism 36 extends to the right and left and assumes a flattened configuration after deployment.

It should be noted at this point that orientational terms such as left and right refer to the subject drawing as viewed by an observer. The drawing figures depict their subject matter in orientations of normal use, which could obviously change with changes in position. Therefore, orientational terms must be understood to provide semantic basis for purposes of description, and do not limit the invention or its component parts in any particular way.

Preferably, the guard 32 and particularly, the hollow cover 34 and the spring mechanism 36 are fabricated monolithically as a single unit from an elastic or resilient material such as a synthetic polymer. This constituency enables the needle device 10 to be manufactured inexpensively by molding techniques, and also enables the various deflections of the guard 32 which are necessary to the functions described herein.

The spring mechanism 36 may assume the construction shown in FIG. 4, to which reference is now made. Whereas the guard 32 has been described in terms of being integrally formed with components of the needle device 10 such as the hollow housing 12, it would also be possible to provide the guard 32 as a separate or stand-alone device which is to be installed on a needle device which is similar to the needle device 10 but lacks the guard 32. For example, a guard such as the stand alone guard 132 shown in FIG. 4 could be commercially available as an accessory for use with otherwise conventional needle devices. Apart from their connection to their respective associated needle devices, such as the needle device 10, the guards 32 and 132 may be essentially similar in structure and function.

The guard 132 may comprise a mount 133 which is dimensioned and configured to engage and couple to a conventional needle device such as the needle device 10 but lacking a device having the function of the guard 32 of FIG. 1 or the guard 132 of FIG. 4. The mount 133 may comprise a sleeve, arm, assembly incorporating plural arms or corresponding members (only the sleeve option is shown), which engages its associated needle device by friction, by threaded connection, by bayonet connection, or in still other ways.

A spring mechanism 136 which is similar in structure and function to the spring mechanism 36 of FIG. 1 may comprise a first relatively rigid link section 135 coupled to the mount 133 and a second relatively rigid link section 137 coupled to a hollow cover 134 which hollow cover 134 may be similar in structure and function to the hollow cover 34 of FIG. 1. A first live hinge 139 may be disposed to couple the first relatively rigid link section 135 to the mount 133. A hinge arrangement to be further described hereinafter may be disposed to couple the first relatively rigid link section 135 to the second relatively rigid link section 137. A second live hinge 141 may be disposed to couple the second relatively rigid link section 137 to the hollow cover 134. Actual connection between the second live hinge 141 and the hollow cover 134 may be accomplished using an intermediate component 143 which is coupled to both the hollow cover 134 and the second live hinge 141.

Description thus far requires that a flexible connection exist between the first relatively rigid link section 135 and the second relatively rigid link section 137. This flexible connection may be provided by a third relatively rigid link section 145 disposed serially between the first relatively rigid link section 135 and the second relatively rigid link section 137. The hinge arrangement incorporated into the priorly described connection may comprise a third live hinge 147 disposed to couple the first relatively rigid link section 135 to the third relatively rigid link section 145, and a fourth live hinge 149 disposed to couple the third relatively rigid link section 145 to the second relatively rigid link section 137.

If desired, and with suitable modification to the above described assembly, the third relatively rigid link section 145 may be omitted, provided that a suitable hinge (not shown) couples the first relatively rigid link section 135 to the second relatively rigid link section 137. Because of the possibility that the third relatively rigid link section 145 may be omitted, the invention may be described in terms of a hinge arrangement which is disposed to couple the first relatively rigid link section 135 to the second relatively rigid link section 137, with the understanding that the hinge arrangement may rely upon the third relatively rigid link section 145 and its associated live hinges 147 and 149, or may alternatively rely on a different arrangement.

It may be mentioned here that the hinge arrangement described above relating to the guard 132 of FIG. 4 may be provided in its essence for the guard 32 of FIGS. 1 and 2. Referring momentarily to FIG. 2, it will be seen that the spring arrangement 36 may include live hinges 39, 47, 49, and 41 formed in the guard 32. All live hinges referred to herein, such as the live hinges 39, 47, 49, and 41 may be formed by a suitable reduction in thickness of the component in which they are formed. Thus it is appreciated that the guard 32 of FIGS. 1 and 2 may function in a manner similar to that of the guard 132 of FIG. 4, the latter merely being described in greater detail herein.

The third relatively rigid link section 145 may comprise a first abutment 151which projects towards the second relatively rigid link section 137. The second relatively rigid link section 137 may comprise a second abutment 153 which projects towards the first abutment 151. The first abutment 151 and the second abutment 153 collectively develop force which urges the hollow cover 134 from the retracted position to the deployed position (in a manner similar to that shown and described with reference to FIGS. 1 and 2) when the spring mechanism 136 is pressed against an environmental surface in a manner which compresses the spring mechanism 132 towards the needle, as is seen in FIGS. 1 and 2.

Returning to FIG. 1, it is seen that the hollow cover 34 comprises a forward end wall 42 bearing an opening 44 for enabling passage of and exposing the needle 28 for use when the hollow cover 34 is in the retracted position. When the hollow cover 34 is in the deployed position shown in FIG. 2, it is possible that forceful careless handling of the needle device 10 will cause the needle 28 to protrude through the opening 44. This may be overcome by the following expedient.

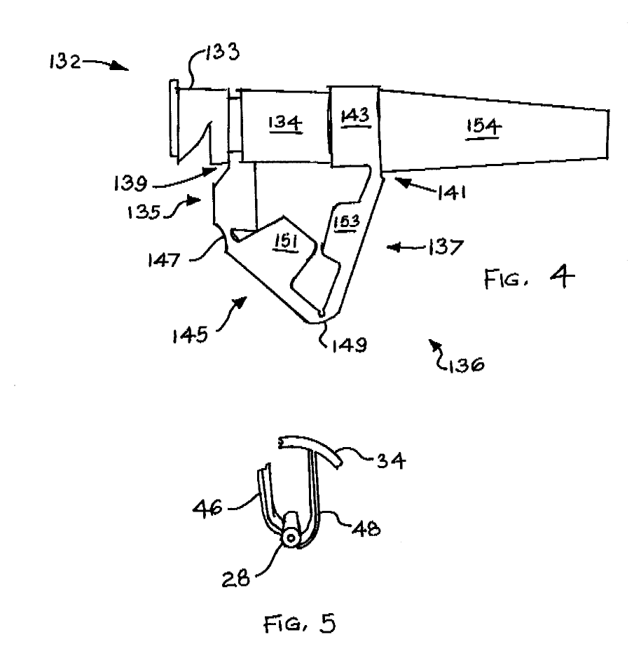

Referring to FIG. 3, when the hollow cover 34 is in the deployed position of FIG. 2, the hollow cover 34 may be maneuvered relative to the needle 28 such that the hollow point 30 of the needle 28 impinges upon the end wall 42 and is thereby precluded from exiting the hollow cover 34 through the opening 44 of the end wall 42. The spring mechanism 32 may be dimensioned and configured and otherwise formed to enable the above described maneuvering.

Again referring to FIG. 2, the guard 32 may comprise a guide disposed between the point of connection of the guard 32 to its associated needle device (such as the needle device 10) and the hollow cover 34. The guide is disposed to engage the needle 28 and maintain the needle 28 in a position generally aligned with the hollow cover 34 when the hollow cover 34 is in the deployed position. The guide may comprise a first hook shaped arm 46 which is mounted to the spring mechanism 36, which projects from the spring mechanism 36 towards the needle 28 when the hollow cover 34 is in the deployed position, and which at least partially encircles the needle 28 when the hollow cover 34 is in the deployed position. The guide may also comprise a second hook shaped arm 48 which is essentially similar to the first hook shaped arm 46, but is preferably formed in mirror image opposition thereto, or alternatively stated, faces in a direction opposite that of the first hook shaped arm 46 relative to the needle 28 when the hollow cover 34 is in the deployed position.

The nature of the first hook shaped arm 46 and the second hook shaped arm 48 is better revealed in FIG. 5.

Returning to FIG. 4, the guard 132 may comprise a cap 154 which is removably engageable with the guard 132 so as to seal the hollow cover 134 against casual contamination by dirt and microbes and the like, and to cover the needle (such as the needle 28) when the hollow cover 134 is in the retracted position. The cap 154 may snap fit to the intermediate component 143 or otherwise removably engage the guard 132. Of course, the guard 32 of FIG. 1 may have a cap (not shown) corresponding in structure and function to the cap 154 of FIG. 4.

Although the invention has been described in terms of certain components being referred to in either the singular or the plural, other arrangements are possible. For example, it is to be understood that due to the conceptual description presented herein, components presented in the singular may be provided in the plural. Illustratively,

The present invention is susceptible to modifications and variations which may be introduced thereto without departing from the inventive concepts. For example the guard 32 may be held in any desired position in order to engage an environmental surface for deployment. Although the force which enables the spring mechanism such as the spring mechanism 36 to operate is shown as being perpendicular to the axis of the needle associated with the spring mechanism, such as the needle 28, it would be possible to arrange the spring mechanism to operate responsive to forces imposed from other directions. It would also be possible to form the spring mechanism which corresponds in function to the spring mechanism 36 in other ways, for example, comprising a single deflectable spring, rather than comprising discrete relative rigid components such as the link arrangement described herein.

While the present invention has been described in connection with what is considered the most practical and preferred embodiment, it is to be understood that the present invention is not to be limited to the disclosed arrangements, but is intended to cover various arrangements which are included within the spirit and scope of the broadest possible interpretation of the appended claims so as to encompass all modifications and equivalent arrangements which are possible.

I claim:

1. A guard for covering sharp points of needle devices and like devices for penetrating a substrate and establishing fluid communication between the needle device and the substrate for transfer of fluids therebetween, comprising:
a mount which is dimensioned and configured to engage and couple to a needle device having a needle having a longitudinal axis and terminating in a sharp point;
a hollow cover for covering the sharp point of the needle, which said hollow cover is adapted to be movable between a deployed position covering the sharp point of the needle and a retracted position which leaves the sharp point of the needle sufficiently exposed to be able to penetrate the substrate; and
a spring mechanism disposed to propel the hollow cover from the retracted position to the deployed position when the guard is urged against an environmental object in a direction which is out of registry with the longitudinal axis of the needle such that force is imposed on the spring mechanism.

2. The guard of claim 1, wherein the mount, the hollow cover, and the spring mechanism are fabricated monolithically as a single unit.

3. The guard of claim 1, wherein the spring mechanism comprises a first relatively rigid link section coupled to the mount and a second relatively rigid link section coupled to the hollow cover, a first live hinge disposed to couple the first relatively rigid link section to the mount, a hinge arrangement disposed to couple the first relatively rigid link section to the second relatively rigid link section, and a second live hinge disposed to couple the second relatively rigid link section to the hollow cover.

4. The guard of claim 3, wherein the spring mechanism comprises a third relatively rigid link section disposed serially between the first relatively rigid link section and the second relatively rigid link section, and the hinge arrangement comprises a third live hinge disposed to couple the first relatively rigid link section to the third relatively rigid link section and a fourth live hinge disposed to couple the third relatively rigid link section to the second relatively rigid link section.

5. The guard of claim 4, wherein the third relatively rigid link section comprises a first abutment which projects towards the second relatively rigid link section, and the second relatively rigid link section comprises a second abutment which projects towards the first abutment, and the first abutment and the second abutment collectively develop force which urges the cover from the retracted position to the deployed position when the spring mechanism is pressed against an environmental surface in a manner which compresses the spring mechanism towards the needle.

6. The guard of claim 1, wherein the hollow cover comprises a forward end wall bearing an opening for exposing the needle for use when the hollow cover is in the retracted position.

7. The guard of claim 6, wherein the spring mechanism is dimensioned and configured to enable the hollow cover to be maneuvered relative to the needle in the deployed position such that the sharp point of the needle impinges upon the end wall and is thereby precluded from exiting the hollow cover through the opening of the end wall.

8. The guard of claim 1, further comprising a guide disposed between the mount and the hollow cover, wherein the guide is disposed to engage the needle and maintain the needle in a position generally aligned with the hollow cover when the hollow cover is in the deployed position.

9. The guard of claim 8, wherein the guide comprises a first hook shaped arm which is mounted to the spring mechanism, which projects from the spring mechanism towards the needle when the hollow cover is in the deployed position, and which at least partially encircles the needle when the hollow cover is in the deployed position.

10. The guard of claim 9, wherein the guide comprises a second hook shaped arm which is mounted to the spring mechanism, which projects from the spring mechanism towards the needle when the hollow cover is in the deployed position, and which at least partially encircles the needle when the hollow cover is in the deployed position, wherein the second hook shaped arm faces in a direction opposite that of the first hook shaped arm relative to the needle when the hollow cover is in the deployed position.

11. The guard of claim 1, further comprising a cap which is removably engageable with the guard so as to seal the hollow cover and cover the needle when the hollow cover is in the retracted position.

12. A needle device for containing a supply of a fluid, penetrating a substrate, and establishing fluid communication between the needle device and the substrate for transfer of fluids therebetween, comprising:
a hollow housing having a proximal end, a distal end, and a fluid chamber disposed between the proximal end and the distal end;
a piston assembly slidably disposed within the fluid chamber, including a piston disposed to exert force on fluid contained within the fluid chamber;
a hollow needle disposed in fluid communication with the fluid chamber, which said hollow needle has a longitudinal axis and terminates in a sharp point capable of penetrating the substrate; and
a guard for covering the sharp point of the needle, wherein the guard comprises
a hollow cover for covering the sharp point of the needle, which said hollow cover is adapted to be movable between a deployed position covering the sharp point of the needle and a retracted position which leaves the sharp point of the needle sufficiently exposed to be able to penetrate the substrate; and
a spring mechanism disposed to propel the hollow cover from the retracted position to the deployed position when the guard is urged against an environmental object in a direction which is out of registry with the longitudinal axis of the needle.

13. The needle device of claim 12, wherein the housing, the hollow cover, and the spring mechanism are fabricated monolithically as a single unit.

14. The needle device of claim 12, wherein the spring mechanism comprises a first relatively rigid link section coupled to the hollow housing and a second relatively rigid link section coupled to the hollow cover, a first live hinge disposed to couple the first relatively rigid link section to the hollow housing, a hinge arrangement disposed to couple the first relatively rigid link section to the second relatively rigid link section, and a second live hinge disposed to couple the second relatively rigid link section to the hollow cover.

15. The needle device of claim 14, wherein the spring mechanism comprises a third relatively rigid link section disposed serially between the first relatively rigid link section and the second relatively rigid link section, and the hinge arrangement comprises a third live hinge disposed to couple the first relatively rigid link section to the third relatively rigid link section and a fourth live hinge disposed to couple the third relatively rigid link section to the second relatively rigid link section.

16. The needle device of claim 15, wherein the third relatively rigid link section comprises a first abutment which projects towards the second relatively rigid link section, and the second relatively rigid link section comprises a second abutment which projects towards the first abutment, and the first abutment and the second abutment collectively develop force which urges the cover from the retracted position to the deployed position when the spring mechanism is pressed against an environmental surface in a manner which compresses the spring mechanism towards the needle.

17. The needle device of claim 12, wherein the hollow cover comprises a forward end wall bearing an opening for exposing the needle for use when the hollow cover is in the retracted position.

18. The needle device of claim 17, wherein the spring mechanism is dimensioned and configured to enable the hollow cover to be maneuvered relative to the needle in the deployed position such that the sharp point of the needle impinges upon the end wall and is thereby precluded from exiting the hollow cover through the opening of the end wall.

19. The needle device of claim 12, further comprising a guide disposed between the housing and the hollow cover, wherein the guide is disposed to engage the needle and maintain the needle in a position generally aligned with the hollow cover when the hollow cover is in the deployed position.

20. The needle device of claim 19, wherein the guide comprises a first hook shaped arm which is mounted to the spring mechanism, which projects from the spring mechanism towards the needle when the hollow cover is in the deployed position, and which at least partially encircles the needle when the hollow cover is in the deployed position.
